(19) United States
(12) Patent Application Publication
(10) Pub. No.: US 2026/0191756 A1
(43) Pub. Date: Jul. 9, 2026

(54) NON-PULVERULENT SOLID COMPOSITION COMPRISING AT LEAST 8% BY WEIGHT OF DISINTEGRANT(S)

(71) Applicant: SOLIFEEL, Marseille (FR)

(72) Inventors: Sophie AGAT, AIX EN PROVENCE (FR); Sébastien BARDON, VEYRIER DU LAC (FR)

(21) Appl. No.: 19/132,402

(22) PCT Filed: Dec. 1, 2023
(86) PCT No.: PCT/EP2023/083926

§ 371 Date: May 23, 2025

(30) Foreign Application Priority Data
Dec. 1, 2022 (FR) 2212636

(51) Int. Cl. A61K 8/73; A61K 8/02; A61K 8/25; A61Q 19/10

(52) U.S. Cl. CPC A61K 8/731; A61K 8/0216; A61K 8/0279; A61K 8/25; A61K 8/732; A61Q 19/10

(57) ABSTRACT

A non-pulverulent solid composition comprising at least 8% by weight of disintegrant(s), as well as associated methods of manufacture.

The invention relates to a non-pulverulent solid composition comprising at least 8% by weight of disintegrant(s).

At the current time, cleaning compositions and cosmetic compositions are generally in liquid or solid form e.g. sticks, compacted or loose powders, pods (or sheets) to be dispersed in water. However, none of these solutions are fully satisfactory.

Liquid products are advantageous, in particular in terms of sensory feeling, texture and, if intended to be applied to keratin material, have comfortable application. On the other hand, they are based on distribution circuits that are ecologically adverse since they are mostly composed of water and therefore of large volume and weight.

With regard to sticks, the use thereof in cosmetics in particular involves repeated contacts between the solid composition and the treated keratin material, which for obvious reasons is hardly hygienic. In addition, degradation of sicks and/or breakage and/or disintegration thereof before they are fully consumed is a source of spoilage and therefore of waste.

With regard to powders, the hydration thereof often leads to the formation of aggregates likely to reduce desired performance and/or comfort on application.

Finally, regarding pods which are generally based on polyvinyl alcohol (PVA or PVOH), these are misleadingly ecological solutions since, when dissolved in water, PVA pollutes groundwater tables. In addition, PVA generally imparts a tacky, unpleasant texture.

Also, solid products, in particular household products, laundry products, soaps and/or shampoos often have insufficient lathering properties, in particular when compared with liquid compositions. In addition, after contacting with an aqueous phase, the periphery of solid compositions such as soaps often develops into a slimy gel-cream texture perceived by users to be unpleasant. This disadvantage can lead users to discard the solid composition, which is questionable from an economic and ecological viewpoint.

Some of these disadvantages argue in favour of solid compositions in the form of single doses. However, it is observed that their size and volume are generally imposed to guarantee easy handling by users, and are generally larger than needed to obtain the desired technical effect. Part of these solid single-dose compositions is in effect superfluous and therefore the source of waste and pollution.

As will become apparent from the example below, the inventors have observed that it is possible to overcome the aforementioned disadvantages by means of a non-pulverulent solid composition of the invention.

The invention therefore concerns a non-pulverulent solid composition having a content of disintegrant(s) higher than or equal to 8% by weight relative to the total weight of the solid composition.

Disintegrants are already used in solid composition, but generally in tablets and capsules based on compacted powders to ensure rapid break-down into their elementary particles, thereby to facilitating the dissolution or release of the active ingredients.

The present invention is unexpected insofar as it is based on the use of dispersant(s) in a non-pulverulent solid composition, and in contents far removed from the recommendations of suppliers and standards of use, generally in the region of 0.5 to 5% by weight of dispersant(s) relative to the weight of the composition containing the same.

As a result, a non-pulverulent solid composition is not in the form of a tablet or capsule containing a compacted powder.

Also, if the solid composition is intended for cleaning a surface, or a keratin material and/or for the care of keratin material, the inventors have observed an unexpected additional effect. Said solid composition, when in contact with an aqueous phase, produces a larger volume of lather and in less time than a solid composition of same size and volume but not containing a dispersing agent or comprising a lower content of dispersant(s).

The invention therefore also allows adjustment of the rate at which the solid composition will swell and/or melt, even lather, by adjusting the content of dispersant(s).

These advantages are all the more surprising since they are not detrimental to the stability, mechanical strength of the solid compositions of the invention and even to the transition kinetics thereof from the original solid state to the liquid state, after contacting with water.

Unless otherwise stated, in the remainder hereof it is considered that temperature is ambient temperature (par e.g. T=25° C.±2° C.) and pressure is atmospheric pressure (760 mm de Hg, i.e. 1.013.105 Pa or 1013 mbar).

By «solid», in the meaning of the present invention, it is meant to designate a composition which, at ambient temperature and atmospheric pressure, is not likely to collapse under its own weight. Advantageously, it is meant to designate any composition having compressive strength higher than or equal to 20 g, preferably higher than or equal to 50 g, and better still higher than or equal to 100 g, at ambient temperature (20-25° C.), after penetration by a cylindrical probe of revolution having a diameter of 0.8 cm into the matrix of the composition to a depth of 1 mm at a rate of 0.5 mm/s and removal of said probe from the matrix of the composition at a rate of 0.5 mm/s, compressive strength being measured with an analyzer of the type “LFRA Texture Analyzer” marketed by STEVENS/MECHTRIC.

A composition of the invention is not in the form of a paste. Nonetheless, at the time of manufacture thereof, in particular if recourse is made to an extrusion method described below, the composition of the invention can be in paste form.

A solid composition of the invention is non-pulverulent, and is therefore in non-particulate form. A composition of the invention is therefore not in the form of a power, in particular in the form of a loose or compacted powder. Finally, a composition of the invention is not a porous solid either derived from the agglomeration of powders.

In a first embodiment, a composition of the invention is not an expanded composition, and is therefore not a foam. In a second embodiment, a composition of the invention can be an expanded composition and can therefore be in the form of a foam.

Advantageously, a solid composition of the invention is not composed of a network of fibres.

Preferably, a composition of the invention does not comprise an alcohol.

Preferably, a solid composition of the invention does not comprise a polyvinyl alcohol (PVA or PVOH), polyacrylate, copolymer of acrylic acid and methacrylic acid, polyvinylidene fluoride (PVDF), polymethyl methacrylate (PMMA), polyvinylpyrrolidone, polyalkylene oxide, polyacrylonitrile (PES or PAN), polymethacrylate, polyethylene terephthalate, polyethylene, polypropylene, nylon, hyaluronic acid, starch and derivatives thereof, pullulan, gelatine, cellulose (e.g. rayon), hydroxypropylmethylcellulose, methylcellulose, carboxymethylcellulose, cotton, and any of the mixtures thereof.

Preferably, a composition of the invention does not comprise (or is not based on) a water-soluble solid substrate e.g. of hydrogel type, in particular starch-based, possibly unstructured, and/or poloxamer 407. However, this does not prevent the composition of the invention from comprising starch as described below.

Advantageously, a solid composition of the invention is not in the form of a mask.

Preferably, a solid composition of the invention is not coated with a water-soluble coating.

Advantageously, a solid composition of the invention is not a pharmaceutical composition.

The solid nature of a composition of the invention can be based on a phase mostly, even fully, composed of a fatty phase comprising at least one lipophilic gelling agent (also called hereafter «solid fatty phase»).

Also, the solid nature can be based on a phase derived from a saponification reaction (or «soap-forming process»), and therefore follow from the conversion of a fatty phase comprising at least one oil, preferably vegetable oil, under the action of sodium hydroxide and optionally heat (also called hereafter «soap phase»).

In addition, the solid nature can be based on a phase comprising at least one synthetic detergent (or surfactant) (also called hereafter «SYNDET phase»). As an illustration it is CETYL ALCOHOL, fully or partially, which contributes towards imparting the solid nature to a SYNDET.

The solid nature of a composition of the invention can therefore be based on (i) a solid fatty phase, (ii) a soap phase, and/or (iii) a SYNDET phase.

In the remainder of the description, the «solid fatty phase», «soap phase» and SYNDET phase can be commonly designated by the expression «solid phase».

Preferably, a solid composition of the invention is single-phase and is therefore formed solely of the solid phase.

In one particular embodiment, a composition of the invention may additionally comprise an aqueous phase dispersed in the solid phase, preferably in encapsulated form e.g. in the form of matrix spheres (or beads), or of core/shell type e.g. (or capsules), for example in the form of capsules as described in WO2010063937.

Preferably, a composition of the invention is a single-dose composition.

This embodiment is particularly advantageous from a hygiene viewpoint in that it avoids repeated contacts between the solid composition and the surface to be treated.

Therefore, the invention also concerns a packaging comprising several solid compositions of the invention in single-dose form, for example of different fragrance, shape, colour and/or different cosmetic effect or active ingredient.

Preferably, a composition of the invention is a topical composition. A composition of the invention is not an oral composition.

In particular, a composition of the invention is not an oral composition or an orodispersible composition.

A solid composition of the invention can be of any shape, e.g. spherical, ellipsoid, tetrahedral, or polygonal, in particular in the form of a parallelogram, preferably a rectangular parallelogram.

Preferably, in particular to ensure easy handling, a solid composition of the invention has:
a width, or diameter, of between 1 cm and 10 cm, preferably between 2 cm and 8 cm, in particular between 3 cm and 6 cm, and better still between 4 cm and 5 cm; and/or
a height (or thickness) of between 0.5 cm and 5 cm, preferably between 1 cm and 4 cm, and in particular between 2 cm and 3 cm.

In one particular embodiment, a composition of the invention may comprise additional patterns differing from the cavities, said patterns possibly being obtained for example by embossing.

In particular a non-pulverulent solid composition of the invention may comprise compounds (or raw materials) in powder form.

### Lipophilic Gelling Agent

In particular, a solid composition of the invention, particularly the solid fatty phase, soap phase and/or SYNDET phase, advantageously comprises at least one lipophilic gelling agent, in particular at least one solid fat preferably chosen from among at least one wax, at least one pasty fat, at least one butter, and mixtures thereof, for example as described in WO2021234135.

Advantageously, a lipophilic gelling agent is a heat-sensitive gelling agent, namely which reacts to heat, and in particular it is a gelling agent solid at ambient temperature and liquid at a temperature higher than 50° C., preferably higher than 60° C., and better still higher than 70° C. Preferably, a heat-sensitive lipophilic gelling agent of the invention has a melting point of between 50° C. and 130° C., and preferably between 60° C. and 120° C.

Persons skilled in the art will take care to choose the lipophilic gelling agent(s) and/or the amount thereof such that the advantageous properties of the solid composition of the invention are not or are not substantially deteriorated by the envisaged addition. These adjustments lie within the general knowledge of skilled persons.

In particular, a solid composition of the invention, in particular the solid fatty phase, may comprise from 10% to 90%, preferably from 20% to 80%, in particular from 30% to 70%, and better still from 40% to 60% by weight of lipophilic gelling agent(s) relative to the total weight of the composition, in particular of the solid fatty phase, soap phase and/or SYNDET phase.

A solid composition of the invention may additionally comprise at least one unsaturated, monounsaturated or polyunsaturated fatty acid, preferably C₆-C₂₂, in particular C₈-C₂₀, and better still C₁₂-C₁₈, e.g. lauric acid (C₁₂), myristic acid (C₁₄), palmitic acid (C₁₆), stearic acid (C₁₈), ricinoleic acid (C₁₈), and mixtures thereof.

A solid composition of the invention, in particular the solid fatty phase, may additionally comprise at least one oil, a vegetable oil in particular e.g. copra oil, coconut oil, olive oil, sweet almond oil, castor oil, Babassu oil, and mixtures thereof. As oils, mention can be made for example of those described in WO2021234135. In particular, a solid composition of the invention may comprise from 10% to 90%, preferably 20% to 80%, in particular 30% to 70%, and better still from 40% to 60% by weight of oil(s) relative to the total weight of the composition.

### Disintegrant

A solid composition of the invention, in particular the solid fatty phase, soap phase and/or SYNDET phase, comprises at least 8% by weight of disintegrant(s) relative to the total weight of the solid composition, even of the solid fatty phase, soap phase and/or SYNDET phase.

By «disintegrant» in the meaning of the invention, it is meant to designate any raw material able to induce, even increase the disintegration rate of a composition comprising the same.

In particular, a disintegrant of the invention can be chosen from among disintegrants which swell in water, promoting penetration of water into the solid composition followed by breakdown of the latter, effervescent disintegrants for which disintegration is ensured via the release of gas which occurs when the solid composition is placed in contact with water, and mixtures thereof.

Disintegrants encompass disintegrants and super-disintegrants.

The disintegrants can be chosen from among sodium starch glycolates; starch, in particular wheat starch or corn starch; pre-gelatinized or partially pre-gelatinized starches; sodium carboxymethyl starch, optionally substituted; starches composed of lactose and corn starch particles; starches composed of pea starch, starches composed of potato starch; starches composed of wheat starch; mannitol-based disintegrants; crystalline cellulose; microcrystalline celluloses or mixture of microcrystalline cellulose with xanthan gum; sodium carboxymethylcellulose (or cellulose gum); calcium croscarmellose; sodium croscarmellose; ethylcellulose; substituted hydroxypropyl cellulose; hydroxypropyl methylcellulose; crosslinked polyvinylpyrrolidone in particular crospovidone (or polyvinylpolypyrrolidone (PVPP); crosslinked polyacrylates; alginic acid and derivatives thereof, for example sodium alginate; and mixtures thereof.

Preferably the disintegrants are chosen from among sodium carboxymethyl starch, sodium croscarmellose, and mixtures thereof.

As disintegrants, the following can be cited for example:
sodium starch glycolates, such as the GLYCOLYS references marketed by Roquette, or Explotab® and Primogel®,
pre-gelatinized or partially pre-gelatinized starches such as the LYCATAB references marketed by Roquette, or C*Pharm® DC 93000 and Starche 1500,
starches composed of lactose and corn starch particles such as the references StarLac and MAIZE STARCH marketed by Roquette,
starches composed of pea starch such as the references ROQUETTE PEA STARCH marketed by Roquette,
starches composed of potato starch such as the POTATO STARCH references marketed by Roquette,
starches composed of wheat starch such as the WHEAT STARCH references marketed by Roquette,
mannitol-based disintegrants such as the reference PEARLITOL marketed by Roquette,
microcrystalline cellulose or mixture of microcrystalline cellulose with xanthan gum such as the MICROCEL references marketed by Roquette or VIVASTAR CS032XV by JRS,
sodium carboxymethylcellulose, such as the reference Blanose CMC 9M31F by Ashland,
sodium croscarmellose such as the reference SOLUTAB marketed by Roquette or the reference VIVASTAR CS40DI by JRS, or the references AC-Di-SOL®, Pharmacel XL; Primellose®, Solutab®, Nymcel ZSX.
crosslinked polyvinylpyrrolidone e.g. those marketed under the trade names Kollidon® CL, Kollidon® CL-M, Polyplasdone® XL, Polyplasdone® XL-10, Polyplasdone® INF-10,
crosslinked polyacrylates e.g. those marketed by ROHM and HAAS under the trade name Acusol, and
mixtures thereof.

A solid composition of the invention, in particular the solid fatty phase, soap phase and/or SYNDET phase, may have a content higher than or equal to 8%, preferably a content higher than or equal to 10%, in particular a content higher than or equal to 12% by weight of disintegrant(s) relative to the total weight of the solid composition, in particular of the solid fatty phase, soap phase and/or SYNDET phase.

Preferably, a solid composition of the invention may comprise between 8% and 50%, preferably between 10% and 40%, in particular between 12% and 30%, and better still between 15% and 20% by weight of disintegrant(s) relative to the total weight of the solid composition.

In particular, a solid composition of the invention has a content higher than 10%, in particular a content higher than 12% by weight of disintegrant(s) relative to the total weight of the solid composition, in particular the solid fatty phase.

### Detergent(s)—Surfactant(s)

Preferably, a solid composition of the invention may additionally comprise at least one detergent. As detergent, mention can be made for example of a surfactant. Therefore, a solid composition of the invention may additionally comprise at least one detergent, preferably chosen from among a cationic surfactant, anionic surfactant, non-ionic surfactant, zwitterionic (or amphoteric) surfactant, and mixtures thereof.

As surfactant able to be used in a solid composition of the invention, particular mention can be made of Sodium cocoyl isethionate, surfactants in the polyglyceryl family e.g. Polyglyceryl-4 laurate, Sulfosuccinates, Alpha olefins, Alkyl glyceryl ether sulfonates, Sodium cocoyl monoglyceride sulfate, Betaine, and mixtures thereof. This is particularly the case if the composition of the invention is a SYNDET (term derived from «synthetic detergent»), also called «soap-free soap» or «dermatological cleansing bar».

### Additional Compounds

A solid composition of the invention may also comprise at least one additional compound differing from the above-mentioned lipophilic gelling agents, oils, disintegrants and detergents.

A solid composition of the invention may additionally comprise powders; glitter; colouring agents chosen in particular from among colouring agents which may or may not be water-soluble or liposoluble, organic or inorganic; materials having an optical effect; liquid crystals, and mixtures thereof; particulate agents insoluble in the fatty phase; fragrances; fillers of organic or mineral type and in particular pigments; preserving agents; humectants; effervescent agents; diluents; disintegration promoters; stabilizers; chelators; emollients; modifiers of pH, of osmotic force and/or modifiers of refractive index etc., or any usual (cosmetic) additive; and mixtures thereof.

A solid composition of the invention may additionally comprise at least one biological/cosmetic active ingredient chosen from among hydrating agents, vitamins, healing agents, depigmenting agents, UV filters, peeling agents, enzymes, antioxidants, active ingredients stimulating the synthesis of dermal and/or epidermal macromolecules, skin relaxants, antiperspirants, soothing agents and/or anti-ageing agents, and mixtures thereof.

If the solid composition of the invention is dedicated to haircare, the composition of the invention may additionally comprise at least one active hair ingredient chosen in particular from among those described in the patent application filed under No FR2206462.

If the solid composition of the invention is intended to be a cleaning product (excluding of keratin material) in particular as household product and/or laundry product, the solid composition of the invention may additionally comprise at least one alkaline compound, a complexing agent, sequestering agent, an enzyme, bleaching agent, activator, optical brightener, polycarboxylate, abrasive particles, descaler, sodium bicarbonate, a hydrotrope, softener, adjuvant or co-adjuvant, preserving agent, electrolyte, effervescent agent, essential oil, fragrance, biocide, disinfectant, colouring agent, glitter, plasticizer, binder, absorbent, foaming agent, masking agent, pH regulator, or any usual cleaning additive and mixtures thereof, and in particular those described in the patent application filed under No FR2206461.

A solid composition of the invention may additionally comprise at least one hardening agent, chosen for example from among sodium lactate, at least one salt and mixtures thereof. Said ingredients have the particular advantage of hardening the solid composition.

A solid composition of the invention may additionally comprise at least one disintegration promoter, e.g. calcium silicate.

A solid composition of the invention may additionally comprise at least one diluent chosen for example from the group formed by microcrystalline cellulose, mannitol, lactose, sodium carbonate, sodium bicarbonate, sodium carbonate, and mixtures thereof.

As microcrystalline cellulose, particular mention can be made of those marketed under the trade names Vivapur® 99, Vivapur® 101, Vivapur® 102, Vivapur® 200, Avicel® PH 101, Avicel® PH 102, Avicel® PH 105, Avicel® PH 200, Tabulose® 101, Tabulose® 102, Tabulose® 250 Vivapur® 12; Vivapur® 20; Vivapur® 301; Vivapur® 302; Avicel® PH 112; Avicel® PH 113; Avicel® PH 301; Avicel® PH 302; Avicel® PH 103.

Preferably, a solid composition of the invention has a content of less than 8%, even less than 5% by weight of diluent(s) relative to the total weight of the solid composition.

Advantageously, a solid composition of the invention does not comprise polyvinyl alcohol (PVA or PVOH), polyvinylidene fluoride (PVDF), polymethyl methacrylate (PMMA), polyacrylonitrile (PES or PAN), polymethacrylate, polyethylene terephthalate, polyethylene, polypropylene, nylon, hyaluronic acid, cotton, and mixtures thereof.

Advantageously, a solid composition of the invention does not comprise alkyl- and/or alkenyl oligoglycosides, olefin sulfonates and/or dextrins, in particular maltodextrin, nucleating agent in particular talc.

Advantageously, the disintegrant is not chosen from among dextrins, in particular maltodextrin.

Advantageously, a solid composition of the invention does not comprise water-insoluble particles chosen in particular from among inorganic insoluble particles, and more particularly from among carbonates, sulfates, silicates, clays, aluminates, phosphates, sulfates, talc, and combinations thereof, in particular as described in GB2459093.

Advantageously a solid composition of the invention does not comprise polyethylene oxide as described in particular in U.S. Pat. No. 4,277,358.

A solid composition of the invention may additionally comprise at least one solvent able to evaporate in full or in part during the manufacturing method, as described below. As solvent, mention can made for example of an apolar or scarcely polar organic solvent e.g. cyclohexane, THF (tetrahydrofuran), and mixtures thereof.

Persons skilled in the art will take care to choose any constituent compounds of a non-pulverulent solid composition of the invention and/or the amount thereof such that the advantageous properties of a composition of the invention are not, or not substantially, deteriorated. Also, skilled persons will ensure that the type and/or quantity of constituent compounds of a non-pulverulent solid composition of the invention are chosen having regard to the required non-pulverulent solid nature of said composition and/or to the manufacturing method thereof. Such adjustments are within the reach of skilled persons.

If the solid composition of the invention is a soap and/or shampoo, the composition advantageously meets at least one of the criteria (or values) from among those defined below:
the composition has a cleansing capacity of between 12 and 22, the highest number indicating the greatest cleaning capacity
the composition has a hardness number of between 29 and 54, the highest number indicating the hardest soap and the lowest number indicating a gentler soap;
the composition has an Emollience value of between 44 and 69;
the composition has a lathering capacity of between 14 and 46, the highest number indicating the largest bubbles and the lowest number indicating smaller bubbles;
the composition has a creaminess value of between 16 and 48;
the composition has an iodine number of between 41 and 70; and/or
the composition has an lodine Number Saponification (INS) of between 136 and 170, preferably between 150 and 165.

The above criteria (or values) and the measuring method thereof lie within the general knowledge of skilled persons.

The solid nature of a composition of the invention advantageously obviates the need for conventional packaging. The packing of a solid composition of the invention can be restricted to a simple paper wrapper.

### Cavity/Cavities

In one particular embodiment, a solid composition of the invention may advantageously comprise at least one cavity such that said composition has porosity greater than or equal to 20%, preferably greater than or equal to 30%, better still greater than or equal to 40%, in particular greater than or equal to 50%, even greater than or equal to 60%, in particular greater than or equal to 70%, and more particularly greater than or equal to 80%, by volume relative to the total volume of the solid composition.

By «porosity», in the meaning of the present invention, it is meant to designate all the voids in a solid body (or material) these voids able to be filled with fluids, water in particular, when the solid composition is placed in the presence of an aqueous solution e.g. water such as tap water, shower or bath water.

By «cavity», in the meaning of the present invention it is meant to designate an empty space inside a solid body and which communicates directly with the outside medium, similar for example to the cavities illustrated in FIGS. 1 to 3 and 6 in the patent application filed under No FR2203881. A cavity can therefore indifferently be designated by the terms «orifice», «pore» or «hole». Advantageously, the cavity/cavities are through cavities i.e. the cavity designates an empty space inside a solid body and which communicates directly with the outside medium either side of the solid body.

Therefore, the volume of the cavity/cavities in a solid composition of the invention represents at least 20%, in particular at least 30%, preferably at least 40%, in particular at least 50%, even at least 60%, in particular at least 70%, and more particularly at least 80%, of the total volume of the solid composition.

Advantageously, a solid composition of the invention has a density of between 0.2 and 0.8, preferably between 0.3 and 0.7, in particular between 0.4 and 0.6.

A solid composition of the invention can have a total volume of between 0.125 cm³ and 100 cm³, preferably between 0.250 cm³ and 75 cm³, in particular between 0.5 cm³ and 50 cm³, better still between 1 cm³ and 25 cm³, and more particularly between 2.5 cm³ and 15 cm³.

A solid composition of the invention can have a weight of between 0.025 g and 200 g, preferably between 0.20 g and 150 g, in particular between 0.5 g and 100 g, better still between 1 g and 75 g, and more particularly between 2.5 g and 50 g, even between 5 g and 25 g («g» corresponding to the unit «gram»).

A solid composition of the invention advantageously has a ratio between the volume of the cavities and the total volume of the composition of between 0.05 and 0.9, preferably between 0.1 and 0.8, in particular between 0.2 and 0.9, preferably between 0.2 and 0.8, better still between 0.2 and 0.7, even between 0.2 and 0.6.

By «total volume», it is meant to designate the volume resulting from the sum of the volume of solid and the volume of the cavities, whether or not the aqueous and/or fatty gelled phase described below is present in all or some of the cavities.

In the present invention, the cavities can be said to be macroporous i.e. having pores of minimum width, even diameter, greater than or equal to 0.5 mm, preferably greater than or equal to 0.75 mm, and in particular greater than or equal to 1 mm, even between 0.5 mm and 2 cm, more particularly between 1 mm and 2 cm, preferably between 2 mm and 1 cm, in particular between 3 mm and 75 mm, better still between 4 mm and 50 mm, and more particularly between 5 mm and 25 mm. In particular, the pores have a minimum width, or diameter, of between 0.5 mm and 5 mm, preferably between 1 mm and 3 mm.

In this respect, the Applicant indicates that, porosity being equivalent, the manufacture of a stable solid composition is:
easy in the presence of cavities of small size e.g. those contained in solid substrates in which porosity results from the insertion of a gas during the manufacturing method, similar to the description given in U.S. Pat. No. 8,349,341, but
much more difficult in the presence of macroporosities such as those under consideration in the present invention.

The presence of cavities having the contents and/or volumes and/or dimensions considered above affords access to solid compositions requiring fewer raw materials whilst maintaining satisfactory handling properties. Said solid composition of the invention is therefore able to generate less loss. This weight gain has the additional advantage of permitting transport that is less polluting and less costly. A solid composition of the invention is therefore advantageous both from an ecological viewpoint and from an economic viewpoint.

In addition, the inventors have observed an unexpected effect in terms of texture and transformative effect when said solid composition of the invention is applied to the surface to be treated. The inventors have effectively observed that a solid composition of the invention exhibits even faster transition kinetics from the original solid state towards a liquid state e.g. of gel or cream type. Without wishing to be bound by any theory, the inventors are of the opinion that the presence of a cavity/cavities in the solid composition allows facilitated transition towards a viscous liquid composition, for example when the solid composition is mixed by hand, or allows an increased contact surface with the aqueous phase, water in particular, and hence better hydration of said solid composition.

The cavity/cavities of a solid composition of the invention can be of any shape e.g. circular, oblong, ovoid, triangular, polygonal in particular hexagonal, of honeycomb or tessellated shape (or partitioned) for example of Voroni diagram type, preferably of honeycomb shape as illustrated in FIGS. 3 and 6 of the patent application filed under No FR2203881.

The cavity/cavities of a solid composition of the invention can also represent letters or the logo of a trademark or company.

A solid composition of the invention comprises one or more cavities, preferably several cavities, the same or different.

In a first variant, a composition of the invention comprises at least one cavity, in particular a through cavity.

In a second preferred variant, a composition of the invention comprises several cavities, through cavities in particular, as illustrated in the patent application filed under No FR2203881.

Advantageously, the cavities are through cavities along an axis x-x′.

In one particular embodiment, a composition of the invention may comprise at least two cavities connected together. Said embodiment is advantageous since it promotes phenomena of capillarity at the time of contact with water, allowing an improvement in the aforementioned technical effects.

Advantageously, a solid composition of the invention comprises several cavities which have a honeycomb structure. This honeycomb structure is particularly advantageous in that it permits the manufacture of a solid composition having a ratio «volume of the cavity/cavities/total volume of the composition» that is very advantageous, in particular higher than or equal to 0.5, even higher than or equal to 0.75, without being detrimental to the handling or robustness of said composition when subjected to the mechanical stresses it is likely to undergo on application and/or during storage. In other words, this honeycomb structure offers an excellent trade-off between cavity volume and mechanical strength.

Advantageously, the cavity/cavities of a solid composition of the invention have walls having a thickness of between 10 μm and 1 500 μm, preferably between 20 μm and 1 000 μm, in particular between 30 μm and 750 μm, more particularly between 50 μm and 500 μm, even between 100 μm and 400 μm. In particular, the cavity/cavities of a solid composition of the invention have walls having a thickness of between 250 μm and 500 μm.

Advantageously, a solid composition of the invention comprises several cavities which take up a honeycomb structure comprising walls having a thickness of between 10 μm and 600 μm, preferably between 20 μm and 500 μm, in particular between 30 μm and 400 μm, more particularly between 50 μm and 300 μm, even between 100 μm and 200 μm.

In one particular embodiment, a solid composition of the invention, in particular when it has a configuration of honeycomb type as previously described, may comprise a plurality of sections in particular polygonal, each section defining a breakable perimeter allowing the separation of at least one section from the remainder of the solid composition. Therefore, with this embodiment, each section of the solid composition corresponds to a single dose.

### Manufacturing Method

A solid composition of the invention can be obtained with any method known to skilled persons and adapted for the manufacture of a solid composition, subject to necessary adaptations to ensure the forming of the cavity/cavities, if any, and hence of meeting the criterion of associated required porosity as previously described.

Preferably, the method for manufacturing a solid composition of the invention does not comprise an aeration/injection step of a gas e.g. air, optionally followed by a forming step and drying.

### Method No 1

In a first variant, a method for manufacturing a composition of the invention comprises at least the steps of:
(i) providing a fatty phase, a phase derived from a saponification reaction and/or a phase comprising at least one synthetic detergent (or surfactant) (i.e. SYNDET phase), in liquid or semi-liquid form and comprising at least 8% by weight of disintegrant(s);
(ii) pouring the first phase into a mould and drying until a solid composition is obtained;
(iii) cutting the solid composition to the desired shape, whereby a solid composition of the invention is obtained.

In one particular embodiment, the manufacturing method according to this first variant may additionally comprise a step (iv) to form at least one cavity as defined above in the solid composition obtained at step (iii).

In one particular embodiment, the method for manufacturing the solid composition is based on a saponification method (or «soap-forming process»).

Step (i) is advantageously performed under heat.

Advantageously, step (iii), and step (iv) if performed, are simultaneous.

Advantageously, steps (ii), (iii), and (iv) if performed, are simultaneous, in which case the mould has a shape adapted to ensure forming of the cavity/cavities.

Step (iv) is advantageously performed by means of a punch.

Method no 1 is advantageous in that the offcuts obtained after step (iii), even (iv) if performed, can be reused for further processing. These offcuts can be reheated and fed back into a subsequent no 1 method at step (i) and/or (ii).

Advantageously, in particular if the composition of the invention is a soap and/or shampoo and therefore comprises a soap phase as previously described, a method of the invention before step (ii) may additionally comprise at least one washing step to remove remaining sodium hydroxide.

### Method No 2

In a second variant, a method for manufacturing a composition of the invention is an extrusion method.

Said manufacturing method of the invention comprises at least the steps of:
(a) providing a fatty phase, a phase derived from a saponification reaction and/or a phase comprising at least one synthetic detergent (or surfactant) (i.e. SYNDET phase), in liquid or semi-liquid form, and comprising at least 8% by weight of disintegrant(s);
(b) passing the phase of step (a) through an extrusion device equipped with a die of shape adapted to form a solid bar;
(c) cutting the bar obtained at step (c), after which a solid composition of the invention is obtained.

In one particular embodiment, the manufacturing method of this second variant may additionally comprise a step (d) to form at least one cavity as defined above in the solid composition obtained at step (c).

Step (a) is advantageously performed under heat.

For obvious reasons, step (b) is performed at a temperature adapted so that the composition is of sufficient viscosity and able (i) to pass through the die and (ii) to maintain the shape imparted by the die. Therefore, and if necessary, the composition injected into the extrusion device is heated to change from a solid state to a pasty state and thereby guarantee the proper conducting of this extrusion step.

Step (d) can be performed using a punch.

Preferably, step (d) is performed with a die having a shape adapted to ensure the forming of the cavity/cavities and the size of associated porosity previously described. Therefore step (d) and step (b) are simultaneous.

As an illustration, an extrusion method of the invention comprises a die such as the one illustrated in FIG. 4 of the patent application filed under No FR2203881.

Advantageously, on leaving the die, the temperature can be lower than ambient temperature, for example lower than or equal to 15° C., even lower than or equal to 10° C.

Step (c) can be performed using a knife or cutting wire such as a butter cutting wire.

Preferably, steps (b) and (c), even step (d), are simultaneous.

Preferably, the method for manufacturing a composition of the invention is the extrusion method no 2 described above.

In one particular embodiment, applicable to methods no 1 and no 2 described above, at step (i)/(a) a fluid composition is provided which may comprise at least one solvent able to evaporate in full or in part at step (ii)/(b), even also at step (iii), and/or (iv) if performed,/(c) and/or (d) if performed.

As solvent, mention can be made for example of an apolar or scarcely polar organic solvent e.g. cyclohexane, THF (tetrahydrofuran), and mixture thereof.

In the above methods No 1 and No 2, when the solid phase of the solid composition comprises at least one fatty phase comprising at least one lipophilic gelling agent, step (i)/(a) may require heating of said composition to a temperature higher than the highest melting point of the lipophilic gelling agents included in said composition.

In one particular embodiment, the extrusion method No 2 can be performed using a 3D printing technique.

### Use

A solid composition of the invention can be used as cleaning product in particular as household product and/or laundry product, or in the field of cosmetics in particular for hygiene, cleansing, enhancement, care and/or make-up of keratin material in particular of the skin and/or hair.

In particular a solid composition of the invention can be dedicated to hygiene, to cleansing and/or to enhancement of a keratin material.

In particular, a solid composition of the invention is dedicated to the hygiene of a keratin material, in particular the skin and/or hair, such as toothpastes for example, deodorants, shower gels, intimate cleansing gels, soaps, shampoos, mouthwashes, hair care products (hair dyes, bleaches), hair conditioning products, cleansing products, shaving and depilatory products e.g. after-shaves, depilatory creams and shaving creams, bath and shower preparations e.g. bath foams, bath oils, and bath salts. Preferably, a solid composition of the invention is a soap and/or shampoo.

Advantageously, a solid composition of the invention is not in the form of a mask.

A solid composition of the invention can also be dedicated to cleaning, in particular as household product and/or laundry product. Therefore, a solid composition of the invention dedicated to cleaning advantageously additionally comprises at least one surfactant and/or at least one detergent.

A solid composition of the invention can also be dedicated to the care and/or make-up of a keratin material, in particular the skin and/or lips.

The solid composition of the invention can be used in the presence of water added to the solid composition before, simultaneously and/or after application thereof to the surface to be treated.

A solid composition of the invention, in particular when it comprises a sufficient quantity of aqueous and/or fatty gelled phase, can also be placed directly in contact with the surface to be treated without requiring the addition of water before, simultaneously and/or after application to the surface to be treated.

In one particular embodiment, the present invention concerns a product comprising, or derived from the contacting between, at least one first solid composition as described above and at least one second liquid composition.

### First Solid Composition

In the meaning of the present invention, the first composition can indifferently be designated «first solid composition» or «solid composition», and corresponds to a solid composition as described above.

### Second Liquid Composition

In the meaning of the present invention, the second composition can indifferently be designated «second liquid composition» or «liquid composition».

In a first variant, the second composition is aqueous.

In a second variant, the second composition is anhydrous.

In a third variant, the second composition is an emulsion. An emulsion can be a direct (i.e. oil-in-water), invert (i.e. water-in-oil) or multiple emulsion, in particular a double emulsion (i.e. water-in-oil-in-water, oil-in-water-in-oil, or oil-in-oil-in-water).

In one particular embodiment, the second composition is an emulsion, preferably a stable, direct emulsion with high concentration of dispersed phase, and in particular it is an emulsion of HIPE type (i.e. «High Internal Phase Emulsion») or Apollonian emulsion.

A second composition of the invention is not in solid form at ambient temperature and ambient pressure i.e. it is able to flow under its own weight.

A second composition of the invention is in liquid form, preferably in gel form.

The viscosity of a second composition or at least the continuous phase thereof when in emulsion form, may vary extensively which allows varied textures to be obtained. Viscosity is measured at ambient temperature and ambient pressure following the method described in WO2017046305.

For obvious reasons, the viscosity of the second composition is adjusted to allow easy dispensing/application of the product of the invention.

In one embodiment, the viscosity of the second composition is advantageously from 5 000 mPa·s to 500 000 mPa·s, preferably from 10 000 mPa·s to 300 000 mPa·s, better still from 25 000 mPa·s to 100 000 mPa·s, and more particularly from 50 000 mPa·s to 75 000 mPa·s, as measured at 25° C. following the above-described method.

Preferably, the second composition can reduce the solidity of the first composition. In particular, the second composition can advantageously comprise at least one raw material able to reduce the solidity of the first composition. This embodiment is advantageous in that it allows the solid texture of the first composition to develop towards a softer texture. The term «transformative effect» can be used.

A product of the invention can advantageously have a «first composition(s)/second composition(s)» weight ratio of between 0.01 and 10, preferably between 0.1 and 5, better still between 0.5 and 1.

In one particular embodiment, the first composition may have high contents of biological/cosmetic active ingredients as described below. The first composition can therefore be termed a «booster».

In the light of the foregoing, a product of the invention is an extemporaneous product i.e. it is the user who, on first use of the product, will place the first composition in contact with the second composition.

The invention therefore also concerns a kit comprising:
at least one first solid composition as previously described;
at least one second liquid composition as previously described; and
optionally, at least one container.

A container can indifferently be designated a «container», «packaging» or «primary packaging».

The invention further concerns use of the kit described above, to obtain a product of the invention.

Preferably, a kit of the invention may additionally comprise at least one application/dispensing means.

If the product is intended for cosmetic use, a kit of the invention may additionally comprise at least one means for application to keratin material, chosen for example from the group formed by spatulas, different brushes, puffs or sponges.

In one particular variant, the kit may comprise several first compositions, the same or different, and/or several second compositions, the same or different, and/or at least one application means.

The invention therefore also concerns a kit comprising several first compositions in the form of single doses comprising cosmetic/biological active ingredients and/or having contents of different cosmetic/biological active ingredients.

Therefore, said kit of the invention advantageously allows easy adapting to the effect sought by users.

For a cosmetic product, this embodiment enables a user to decide on the desired cosmetic effect, in particular according to frame of mind (mood), dress style and/or time of day.

### EXAMPLE—COMPARATIVE STUDY FOR CLEANSING OF THE SKIN

### Step 1—Production of the Soap Phase:

Using a mixer and hot water bath at 80° C., the raw materials described in following Table 1 are mixed until a homogeneous mixture is obtained.

| | 1 | 2 | 3 |
|---|---|---|---|
| | (Comp.) | (Comp.) | (Inv.) |
| Raw materials | Weight (%) | | |
| SOAP NOODLES | q.s.* | | |
| (mixture of shea butter, copra oil, olive oil, | | | |
| sunflower seed oil)** | | | |
| COCOA BUTTER | 1.5 | | |
| SODIUM CROSCARMELLOSE | 0 | 5 | 10 |

| | |
|---|---|
| GLYCERINE | 18 |
| Total | 100 |

*in sufficient amount
**fats used for the saponification reaction

### Step 2—Extrusion:

The mixture obtained at step 1 is injected into an extruder equipped with a die as illustrated in FIG. 5 in the patent application filed under No FR2203881. If necessary, the injected composition is previously heated so that it changes from a solid state to a pasty state to guarantee proper conducting of the extrusion step. On leaving the die, the extruded soap bars are cut to obtain single-dose soaps having a thickness of about 4 mm.

### Step 3—Application:

Step 3 was conducted with a panel of 18 women and 18 men aged between 22 and 47 years (hereafter the «candidates»), 3 groups being formed with each group comprising 6 women and 6 men (groups A, B and C).

For each group:
3.1/ The candidates poured a glass filled with 30 cl of tap water onto both hands.
3.2/ The candidates washed their hands with soap 1, 2 or 3, with rotational rubbing of hands containing the tested soap.
3.3/ The candidates rinsed their hands with tap water then dried their hands.

Each of the groups A, B et C used soaps 1, 2 and 3, but each time in different order.

The candidates assessed the performance of soaps 1, 2 and 3 in terms of (i) mechanical strength on handling (ii) solid->liquid transition kinetics at step 3.2/above, (iii) sensory feel and comfort on application, (iv) lathering capacity, and (v) state of soap after use.

### Scoring Criteria:

| SCORING CRITERIA | 0 | 1 | 2 |
|---|---|---|---|
| Mechanical strength | Satisfactory | Moderate | Poor |
| solid -> liquid | <30 seconds | 30-60 seconds | >60 seconds |
| transition kinetics | | | |
| Sensory feel and | Satisfactory | Moderate | Poor |
| comfort on application | | | |
| Lather | High | Moderate | Insufficient |
| State of soap | Fully | Some residues | General structure |
| after use | disappeared | | retained |

The averages of the results obtained with the three groups A, B et C are given in the table below, it being pointed out that the same trends were observed between the three groups.

| | Soap 1 | Soap 2 | Soap 3 |
|---|---|---|---|
| | (Comp.) | (Comp.) | (Invention) |
| Mechanical strength | 0 | 0 | 0 |
| solid -> liquid | 2 | 1 | 0 |
| transition kinetics | | | |
| Sensory feel and comfort on | 1 | 1 | 1 |
| application | | | |
| Lather | 1 | 1 | 0 |
| State of soap after use | 2 | 1 | 0 |

All the tested solid compositions gave satisfactory performance in terms of handling.

Soap 3 of the invention is the one which exhibited the best performance in terms of transition kinetics, the amount of lather formed, and the only one to have been fully used after use and therefore the only one which did not form any residue after use.

A study similar to the one described above was carried out with a SYNDET (also called «soap-free soap» or «dermatological cleansing bar») having the composition given in the table below. Identical results were obtained.

| | 1 | 2 | 3 |
|---|---|---|---|
| | (Comp.) | (Comp.) | (Inv.) |
| Raw material | Weight (%) | | |
| GLYCERINE | q.s.* | | |
| SODIUM COCOYL ISETHIONATE | 40 | | |
| SODIUM CARBOXYMETHYL STARCH | 5 | | |
| SODIUM CROSCARMELLOSE | 0 | 5 | 10 |

| | |
|---|---|
| CETYL ALCOHOL | 13 |
| POLYGLYCERYL-4 LAURATE | 2 |
| Total | 100 |

*in sufficient amount

What is claimed is:

1. A non-pulverulent solid composition having a content of disintegrant(s) higher than or equal to 8% by weight, relative to the total weight of the solid composition.

2. The solid composition according to claim 1, wherein the disintegrant is chosen from among sodium starch glycolates; pregelatinized or partially pregelatinized starches; sodium carboxymethyl starch; starches composed of lactose and corn starch particles; starches composed of pea starch; starches composed of potato starch; starches composed of wheat starch; mannitol-based disintegrants; crystalline cellulose; microcrystalline celluloses or mixture of microcrystalline cellulose with xanthan gum; sodium carboxymethyl cellulose; calcium croscarmellose; sodium croscarmellose; ethyl cellulose; substituted hydroxypropyl cellulose; hydroxypropyl methyl cellulose; crosslinked polyvinylpyrrolidone; crosslinked polyacrylates; alginic acid and derivatives thereof e.g. sodium alginate; and mixtures thereof.

3. The solid composition according to claim 1, wherein the composition additionally comprises at least one diluent.

4. The solid composition according to claim 3, wherein the composition has a content of diluent(s) of less than 8%, by weight, relative to the total weight of the solid composition.

5. The solid composition according to claim 1, wherein the composition additionally comprises at least one disintegration promoter.

6. The solid composition according to claim 1, wherein the composition comprises at least one cavity such that the composition has porosity greater than or equal to 20% by volume, relative to the total volume of said solid composition.

7. The solid composition according to claim 6, wherein the cavity/cavities are through cavities.

8. The solid composition according to claim 6, said composition having one or more cavities the same or different.

9. The solid composition according to claim 6, wherein the shape of the cavity/cavities is circular, oblong, ovoid, triangular, polygonal, honeycomb, or of tessellated shape (or partitioned).

10. The solid composition according to claim 6, wherein the ratio between the volume of the cavity/cavities and the total volume of the composition is between 0.2 and 0.9.

11. The solid composition according to claim 1, wherein said composition additionally comprises at least one detergent.

12. The solid composition according to claim 1, wherein said composition is a single-dose composition.

13. The solid composition according to claim 1, wherein said composition is a topical composition.

14. A method for manufacturing a solid composition according to claim 1, comprising at least the steps of:
(i) providing a fatty phase, a phase derived from a saponification reaction and/or a phase comprising at least one synthetic detergent, in liquid or semi-liquid form, and comprising at least 8% by weight of disintegrant(s);
(ii) pouring the first phase into a mould and drying until a solid composition is obtained;
(iii) cutting the solid composition to the desired shape, whereby a solid composition is obtained according to claim 1.

15. A method for manufacturing a solid composition according to claim 1, comprising at least the steps of:
(a) providing a fatty phase, a phase derived from a saponification reaction and/or a phase comprising at least one synthetic detergent, in liquid or semi-liquid form, and comprising at least 8% by weight of disintegrant(s);
(b) passing the phase of step (a) through an extrusion device equipped with a die having a shape adapted to form a solid bar;
(c) cutting the bar obtained at step (c), after which a solid composition is obtained according to claim 1.

16. A kit comprising:
at least one first solid composition according to claim 1;
at least one second liquid composition; and
optionally at least one container.

17. The solid composition according to claim 3, wherein the at least one diluent is chosen from the group formed by microcrystalline cellulose, mannitol, lactose, sodium carbonate de sodium, sodium bicarbonate, calcium carbonate, and mixtures thereof.

18. The solid composition according to claim 5, wherein the at least one disintegration promoter is calcium silicate.

19. The solid composition according to claim 11, wherein the at least one detergent is chosen from among a cationic surfactant, anionic surfactant, non-ionic surfactant, zwitterionic or amphoteric surfactant, and mixtures thereof.
